(19) United States
(12) Patent Application Publication
(10) Pub. No.: US 2020/0020445 A1
(43) Pub. Date: Jan. 16, 2020

(54) TELEMEDICINE SYSTEM FOR REMOTE MONITORING AND MANAGING PATIENTS UNDERGOING PERITONEAL DIALYSIS AND METHODS EMPLOYED THEREOF

(71) Applicant: SHIVANAND NAYAK KAROPADI, Hyderabad (IN)

(72) Inventor: SHIVANAND NAYAK KAROPADI, Hyderabad (IN)

(21) Appl. No.: 16/348,876

(22) PCT Filed: May 10, 2017
(86) PCT No.: PCT/IB2017/052724

§ 371 Date: May 10, 2019

(30) Foreign Application Priority Data
Mar. 22, 2017 (IN) 201741009938

(51) Int. Cl. G16H 40/67; A61B 5/00; A61M 1/16; G16H 10/60

(52) U.S. Cl. CPC G16H 40/67; A61B 5/7445; A61M 1/28; G16H 10/60; A61M 1/1601

(57) ABSTRACT

Exemplary embodiments of the present disclosure are directed towards a method for remote monitoring and managing patients undergoing peritoneal dialysis by establishing a communication link between a peritoneal dialysis machine; a patient's display device; and a PD nurse's display device and a physician's display device and further communicating a preparatory tutorial for an upcoming peritoneal dialysis procedure to the patient and the PD nurse through the patient's display device and the PD nurse's display device post authentication of the patient and displaying physiological data and symptoms data of the patient undergoing peritoneal dialysis on the patient's display device and the PD nurse's display device and further prescribing a treatment for the patient undergoing peritoneal dialysis by the concerned physician through the physician's display device to the patient's display device; and the PD nurse's display device and performing peritoneal dialysis procedure as per the step by step preparatory tutorial and communicating any contingency to the physician in real time; further monitoring the essential parameters for analysing the peritoneal dialysis procedure; recording the results obtained during the peritoneal dialysis procedure wherever

### TECHNICAL FIELD

The present disclosure generally relates to the field of patient communication tools. More particularly, the present disclosure relates to a distant communication system for tracking the patients undergoing peritoneal dialysis as a preventive care in real time.

### BACKGROUND

Patients on dialysis require ongoing supervision by nephrologists to adjust the dialysis prescription, pre-empted and manage complications. Since peritoneal dialysis (PD) patients normally perform the treatment themselves in their place of residence, they tend to have more autonomy than those treated with haemodialysis, who are required to travel to a health-care facility at least thrice a week to receive the treatment. Patients residing in remote areas may not have a well monitored care as it may not be possible for the nephrologist or the clinical staff to personally monitor the process for PD followed by the patient or his PD nurse. Catheter related infections (CRIs) have become a prominent morbidity factor in PD. Peritonitis and catheter exit-site infections (ESIs) are important causes of hospitalization and catheter loss in patients undergoing chronic peritoneal dialysis. Peritonitis remains a major cause representing technique failure leading to morbidity and mortality in PD patients. Infections are the leading causes of morbidity and one of the leading causes of mortality in the dialysis population. Proper hand hygiene techniques need to be well taught and persistently enforced by patients and medical staff. Studies comparing outcomes of chronic kidney disease patients have consistently shown poorer results in rural than in urban patients.

Patients afflicted with chronic kidney disease and Peritoneal Dialysis (PD) patients, both Manual PD and Automated PD (APD) in particular need to be in regular contact with their doctors and nurses for ongoing monitoring to ensure adequate dialysis, and need to be regularly assessed for complications. Furthermore, the PD patients and their care givers need to be educated on the importance of infection control and an appropriate medical regimen must be established to ensure proper PD delivery, efficacy and safety. The current framework for PD care and monitoring is labour intensive, susceptible to errors and prone to delays. The delivery of care typically consists of manual activities and hand offs including phone calls, site visits, and coordination and communication between teams of professionals such as Nephrologist, PD nurse, dietitian, social worker etc. Therefore the costs and cycle times associated with delivering effective, timely, quality care are significant.

In the light of aforementioned discussion there exists a need for a PD system with proper remote monitoring that can emerge as a safe, viable mode of renal replacement therapy for renal failure patients by minimizing the perceived distance between the patient and the Nephrology team.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards a telemedicine system for remote monitoring and managing patients undergoing peritoneal dialysis and methods employed thereof.

Another exemplary objective of the present subject matter is directed towards a continuous, coordinated, timely, and quality peritoneal dialysis care and monitoring.

Another exemplary objective of the present subject matter is directed towards a step by step comprehendible guide for the patient or the PD nurses to follow.

Another exemplary objective of the present subject matter is directed towards leveraging the availability of ubiquitous wireless access compatible with simple as well as complicated computing devices.

Another exemplary objective of the present subject matter is directed towards automated recording and sharing of the patient information in real time.

Another exemplary objective of the present subject matter is directed towards the flexibility of the user in movement or activities while undergoing peritoneal dialysis.

Another exemplary objective of the present subject matter is directed towards simple and intuitive alarm systems with a high degree of specificity.

Another exemplary objective of the present subject matter is directed towards a peritoneal dialysis system which is non-intrusive, portable and stand alone.

Another exemplary objective of the present subject matter is directed towards a cloud for storing the patient's data and allied information to be retrieved at the user's free will.

Another exemplary objective of the present subject matter is directed towards quality of life parameter to analyze the overall wellness of the patient undergoing PD.

Another exemplary objective of the present subject matter is directed towards a sequential follow up and response to corrective measures.

Another exemplary objective of the present subject matter is directed towards detection of peritonitis, exit site infection, and fluid overload by images and videos with font background.

Another exemplary objective of the present subject matter is directed towards corrective measures

Yet another exemplary objective of the present subject matter is directed towards a patient centric preventive medical care.

Another exemplary aspect of the present subject matter is directed towards establishing a communication link between: a peritoneal dialysis machine; a patient's display device; a PD nurse's display device and a physician's display device.

Another exemplary aspect of the present subject matter is directed towards communicating a preparatory tutorial for an upcoming peritoneal dialysis procedure to the patient and the PD nurse for easy facilitation of the PD procedure.

Another exemplary aspect of the present subject matter is directed towards displaying the patient's physiological data and symptoms data in the respective devices of the PD nurse and physician.

Another exemplary aspect of the present subject matter is directed towards communicating an advice for the patient undergoing peritoneal dialysis procedure by the concerned physician.

Another exemplary aspect of the present subject matter is directed towards and communicating any contingency to the physician in real time.

Another exemplary aspect of the present subject matter is directed towards monitoring the parameters for analyzing the peritoneal dialysis procedure.

Another exemplary aspect of the present subject matter is directed towards recording the test results obtained during the peritoneal dialysis procedure wherever applicable and delivering the results obtained to the physician's display device.

Another exemplary aspect of the present subject matter is directed towards storing the results obtained during the course of the peritoneal dialysis procedure in the cloud for at least one of: instant reference or for future use.

Another exemplary aspect of the present subject matter is directed towards checking for at least one of: exit site infections; clarity of drain bag; and pedal edema post the conclusion of the peritoneal dialysis procedure by the physician.

Another exemplary aspect of the present subject matter is directed towards analyzing of the observations and readings obtained post the peritoneal dialysis procedure and recommendation of prescriptions accordingly.

Another exemplary aspect of the present subject matter is directed towards multiple interconnected devices of the PD nurse, patient and the physician.

Another exemplary aspect of the present subject matter is directed towards a zoom tool embedded in the physician's display device configured to zoom in and zoom out at least one of: the plurality of videos; and a plurality of images.

Another exemplary aspect of the present subject matter is directed towards a cloud of network configured to communicate with the patient's display device; the PD nurse's display device; and the physician's display device to store; recover; and back up the patient's physiological data; and patient's symptoms data.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a block diagram depicting an environment of a telemedicine system for remote monitoring and managing patients undergoing peritoneal dialysis, according to an exemplary embodiment of the present disclosure.

FIG. 2 is a screenshot depicting the login page of the patient undergoing peritoneal dialysis, according to an exemplary embodiment of the present disclosure.

FIG. 3 is a screenshot depicting the preparatory page for the patient undergoing peritoneal dialysis, according to an exemplary embodiment of the present disclosure.

FIG. 4 is a screenshot, depicting the connectivity of the drain to the peritoneal cavity of the patient undergoing PD, according to an exemplary embodiment of the present disclosure.

FIG. 5 is a screenshot, depicting the parameters involved in PD exchange doses for judgement of peritoneal dialysis results, according to an exemplary embodiment of the present disclosure.

FIG. 6 is a screenshot depicting the image upload option by the patient, according to an exemplary embodiment of the present disclosure.

FIG. 7 is a screenshot depicting the physician's dashboard reflecting the wellness pie chart, according to an exemplary embodiment of the present disclosure.

FIG. 8 is a screenshot depicting the diagnosis page of the patient undergoing peritoneal dialysis, according to an exemplary embodiment of the present disclosure.

FIG. 9 is a screenshot depicting the lab reports of the patient undergoing peritoneal dialysis, according to an exemplary embodiment of the present disclosure.

FIG. 10 is a screenshot depicting a graphical representation of prescription details of the patient undergoing peritoneal dialysis, according to an exemplary embodiment of the present disclosure.

FIG. 11 is a screenshot depicting the adequacy of PD dose prescribed for the patient undergoing peritoneal dialysis, according to an exemplary embodiment of the present disclosure.

FIG. 12 is a screenshot depicting vital parameters of the patient undergoing peritoneal dialysis, according to an exemplary embodiment of the present disclosure.

FIG. 13A is a screenshot depicting the inputs regarding the quality of life by the patient undergoing peritoneal dialysis, according to an exemplary embodiment of the present disclosure.

FIG. 13B is a screenshot depicting the inputs regarding the quality of life by the patient undergoing peritoneal dialysis as a histogram, according to an exemplary embodiment of the present disclosure.

FIG. 14 is a screenshot depicting the exit site of the patient undergoing PD, according to an exemplary embodiment of the present disclosure.

FIG. 15 is a screenshot depicting the usage of font sheet for the drain bag of the patient undergoing peritoneal dialysis, according to an exemplary embodiment of the present disclosure.

FIG. 16 is a screenshot depicting the usage of zoom tool for determining pedal edema, according to an exemplary embodiment of the present disclosure.

FIG. 17 is a flow chart depicting a method for remote monitoring and managing patients undergoing peritoneal dialysis, according to an exemplary embodiment of the present disclosure.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

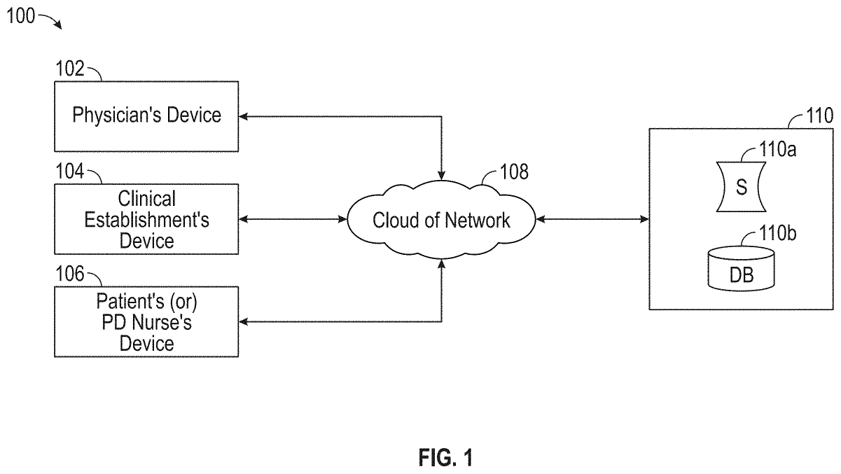

Referring to FIG. 1 is a block diagram 100, depicting an environment of a telemedicine system for remote monitoring and managing patients undergoing peritoneal dialysis, according to an exemplary embodiment of the present disclosure. A physician's device 102 utilised by a physician, a clinical establishment's device 104 operated by a clinical establishment and a patient's and/or PD nurse's device 106 utilised by the patient and/or PD nurse are connected to a system 110 through a cloud of network 108. The system 110 further comprises of a server 110a and a database 110b. The physician may be a general physician, nephrologist, paediatric nephrologist and the like without limiting the scope of the disclosure. The physician would be the one with personal practice and/or a part of clinical establishment. The PD nurse may not be limited to, a nurse, a peritoneal dialysis (hence forth referred to as PD) nurse, a ward boy and the like. The patient referred to, is the patient undergoing PD. The clinical establishment may not be limited to, a hospital, a clinic, a dispensary, a dialysis centre and the like. The physician's device 102, the clinical establishment's device 104 and patient's or PD nurse's device 106, may not be limited to, mobile phones, smartphones, tablets, laptops/desktops, servers, storage, network (wired and wireless) devices and industry standard communication protocols. The devices 102, 104 and 106 along with any necessary peripherals (USB, mouse, monitors, audio jacks etc.) and inherent capabilities in these devices (such as voice, audio, GPS, playback/recording, texting, messaging, email, wireless connectivity, touch screen, camera, Bluetooth, and the like) are inter-connected and integrated into the PD remote software with the specific purposes of helping a PD patient link and communicate in real time with the team of medical professionals, social worker, dietitian, and the like to ensure compliance and adherence to the treatment regimen, enable the patient to ask questions and receive medical advice and allow the team of medical professionals and the PD patients manage their chronic disease condition. The invention leverages the availability of ubiquitous wireless broadband access, ownership of affordable yet extremely versatile and powerful devices such as the smartphones, tablets, iPad and other mobile devices, also Laptops, Desktops and technologies such as cloud computing, social networks etc. The PD remote application (herein referred to as an application) may be a part of the system 110. The application acts as a source of communication between the patient and/or PD nurse, physician and clinical establishment.

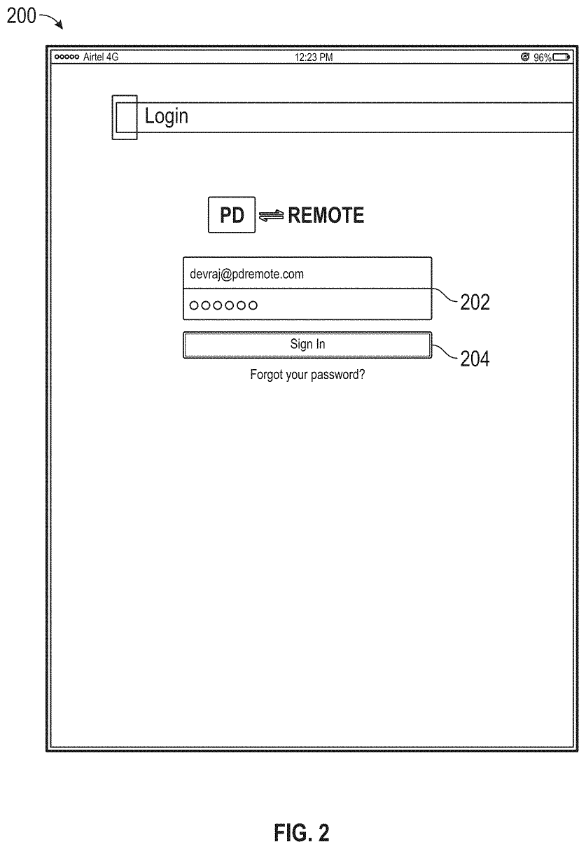

Referring to FIG. 2 is a screenshot 200, depicting the login page of the patient undergoing PD, according to an exemplary embodiment of the present disclosure. The login technically needs a unique ID which is Unique Device Identifier (UDID) of the device being used for the patient either by the patient and/or PD nurse. The UDID is shared to the application developer who creates a link for the patient and/or PD nurse and this link is shared to the patient and/or PD nurse via not limiting to, an e-mail. The Login name and and/or User ID 202 is assigned by the physician in charge for the corresponding patient which the patient can access upon clicking on to the link provided to the patient via an e-mail. The login ID 202 may be used by the patient to click on sign in 204 to access one's account. The link to be created via UDID ensures that the patient's data is safe and is not retrievable in each and every device. This avoids compromise with privacy and also unnecessary sharing of details with multiple physicians avoiding by passing the physician in charge. This also ensures that if PD nurse meddles with the records of the patient this comes to the notice of the physician immediately enabling one to take necessary corrective action.

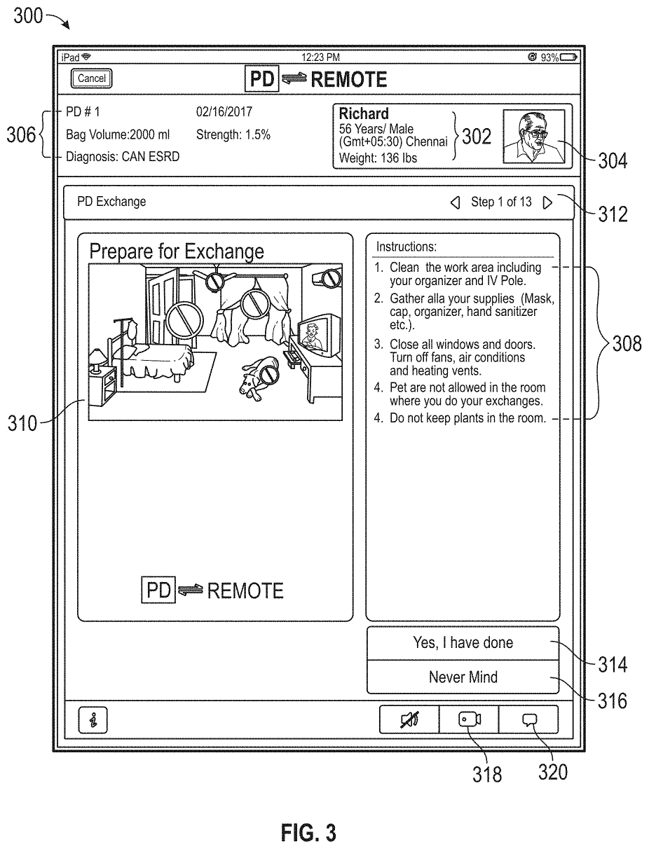

Referring to FIG. 3 is a screenshot 300, depicting the preparatory page for the patient undergoing PD, according to an exemplary embodiment of the present disclosure. The step by step PD procedure, not limited to 13 steps (inclusive of 6 steps of standard hand wash by World Health Organization), is depicted in the application post login by the patient and/or PD nurse. The tutorial acts as a comprehensive aid for home based PD, training inclusive of audio-visual guide and may be used in, not limited to, standalone software for Continuous Ambulatory Peritoneal Dialysis (CAPD) and/or Ambulatory Peritoneal Dialysis (APD). CAPD is manual and PD is automated and/or manual. In the screen shot 300, post login, the basic details 302 of the patient undergoing PD is reflected. The basic details 302 may be inclusive of, but not limited to, name of the patient, age, gender, time zone and place where the patient resides and undergoes PD procedure and body weight. A picture 304 of the patient undergoing PD is displayed. This helps in identifying the patient, more so if there are multiple patients with same name. The details of PD 306 not limiting to, the PD number (PD#1) i.e. the peritoneal dialysis number which the patient is undergoing for the day, the PD bag volume and diagnosis {(End-Stage Renal Disease (ESRD)} are also displayed. The set of instructions 308 before the commencement of PD are displayed. The instructions may be inclusive of, but not limited to, the basic cleansing instructions on surrounding areas, organization of the essential equipment, refraining plants and animals at the vicinity and the like. The exchange must be conducted in the right sequence draining out; flushing before fill and then draining. The pictorial representation of the instructions 310 is displayed for the easy comprehension of the patient and/or PD nurse. The instructions may also be inclusive of advising the patient and/or the PD nurse to check whether there is enough supply of the substances for the PD procedure. For example: if the patient undergoing PD resides in a remote village it is advisable to have the supplies stocked up for a fortnight minimum. The step number is depicted as 312 in the screenshot. Once the instructions are taken and the patient and/or PD nurse is convinced to have followed all the steps, Yes, I have done 314 option may be clicked. If the patient and/or PD nurse is used to these instructions then a never mind 316 option may be chosen. A video and/or image 318 may be uploaded by the patient and/or PD nurse or a message 320 in case of any apprehension and/or need to share a requirement with the physician. The video and/or image 318 and the message 320 are configured to be shared with the physician in real time before, during and after completion of the PD procedure.

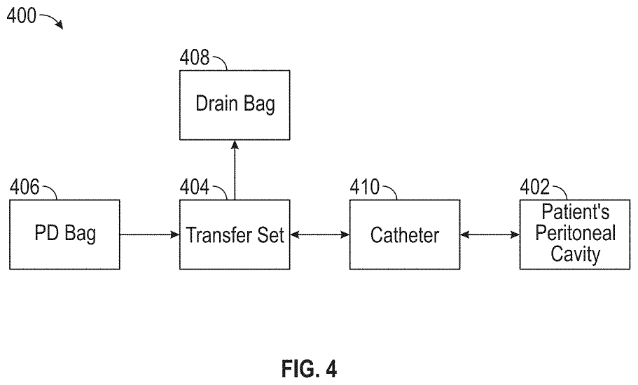

Referring to FIG. 4 is a screenshot 400, depicting the connectivity of the drain to the peritoneal cavity of the patient undergoing PD, according to an exemplary embodiment of the present disclosure. The instructions pertaining to the drain bag attach importance in cases of PD. It is mandatory to check the PD sequence which goes as: draining the waste fluid from the patient's body; flushing out the air bubbles in the transfer tube; and filling of the dialysis fluid in the patient's peritoneal cavity. After having fixed the entire system for PD, not limiting to, the PD bag 406 is connected to the transfer set 404. The fluid line and the drain line are clamped using the clamp which is used externally on the fill line and the drain line to lock the flow of the fluid. Once the drain is completed, the transfer set 404 is locked and the clamp on the fluid line is opened, so that the fluid from the PD bag 406 flushes the fluid line removing the air bubbles in the fluid line. Once the flush is completed then the drain line is locked and the transfer set is opened so that the fluid fills into the patient's peritoneal cavity 402. Once the fill is completed the twist of the transfer set 404, the clamp can be locked and the line can be disconnected, and the transfer set 404 can be closed with the transfer set cap. Till the next exchange the fluid will be in the patient's peritoneal cavity 402 and this time is called dwell time which refers to a single complete PD cycle comprising an aggregate of a drain time, a flush time, and a fill time. The cycle continues as begin of drain, flush and fills. Attaching the drain bag 408 to the patient's peritoneal cavity 402 through a not limiting to, a titanium adaptor is connected with a transfer set 404 (which needs to be changed, not limiting to, once in every four months) which connects the catheter 410 attached to the patient's peritoneal cavity 402. The catheter 410 is the connection between the transfer set 404 and the drain bag 408. A PD bag 406 which contains the PD solution for the exchange is connected to the transfer set 404. It is to be noted, that from hygiene point of view the PD bag 406 and the drain bag 408 may be disposable and for one time use. The connection of transfer set 404 to the drain bag 408 is also an important step forming a part of the training. Once the fixation is appropriate the two white clamps on the catheter 410 may be closed. The PD catheter is surgically inserted in the peritoneal wall of the patient undergoing PD. The transfer set 404 is connected to the catheter 410 inserted inside the patient's peritoneal cavity 402 through a titanium adaptor which is used to join and lock the catheter end with the transfer set 404. The catheter 410 is made of silicone and/or polyurethane with radiopaque strips. The transfer set 404 is made of PVC with flow control and locking mechanisms used to connect the PD bag 406 for the exchange. The transfer set 404 comprises of clamps used externally on the fill line and the drain line to lock the flow of the fluid with transfer set cap where one line is the drain line connected to the drain bag 408 and the other line is the fluid line connected to the PD bag 406. The PD bag 406 may also have a provision for an inject port through which the requisite drug based on the requirement of the patient undergoing PD may be added through a syringe. For example: the external part of the inject port may be surrounded by a polyvinyl cap which has latex in the centre so that upon injection the pierced latex can come back to normal configuration thus avoiding leakage of the PD fluid in the PD bag 406.

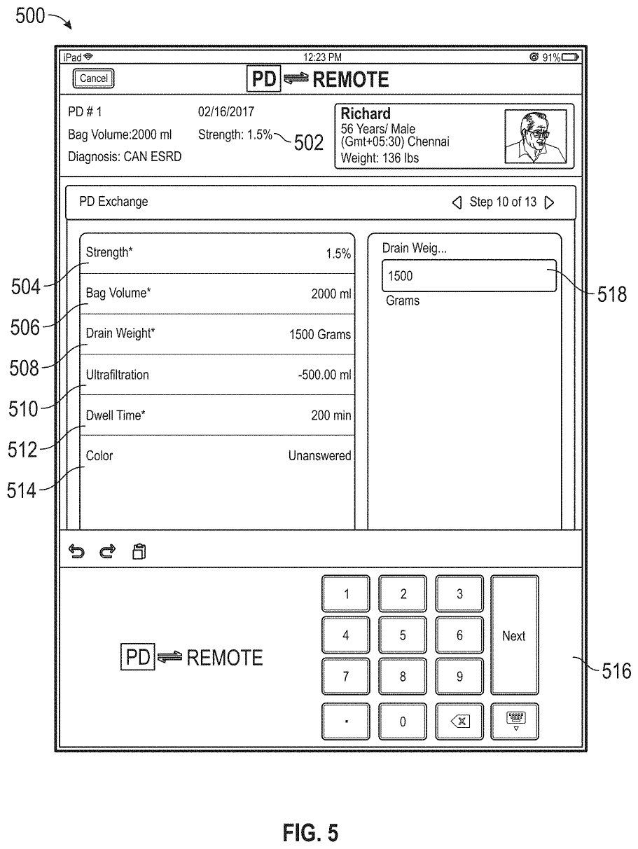

Referring to FIG. 5 is a screenshot 500, depicting the parameters involved in PD exchange doses for judgement of PD results, according to an exemplary embodiment of the present disclosure. The PD exchange-dose 502 is a crucial parameter in analyzing the efficiency of PD being conducted. The parameters which form a part of PD exchange dose may not be limited to, strength of the PD solution 504, PD bag volume 506, drain weight 508, ultrafiltration 510, dwell time 512 and color 514. The values may be fed in column 518 with the help of an inbuilt numeric input device 516. The strength 504 of the PD solution may be depicted as a percentage (%). For example low concentration, medium concentration and high concentration could be that of dextrose solution based on the patient's requirement. The strength 502 (1.5% in the figure which is a low strength). Similarly the PD bag volume 506 (2000 ml in figure) is the volume of the dialysis solution in the bag. The drain weight 508 (1500 ml in figure) is normally measured in a weigh scale. For example: for a patient undergoing PD if the volume of dialysis fluid ingested is 2000 ml, ideally more than 2000 ml should be drained out of the body else, it may be concluded that the patient may be having medical issues. Ultrafiltration 510 (−500.00 ml in the figure) is the amount of peritoneal fluid retained by the patient. The figures enable the physician to be alerted in case of fluid retention so that the physician may fix the prescription accordingly. A dwell time 512 (220 min in the figure) refers to a single complete PD cycle comprising an aggregate of a drain time, a flush time, and the time till the next drain time starts and fill time. It is after the dwell time 512 that the drain weight 508 is measured. The color 514 is another parameter to analyze the PD process. If the color is, not limiting to, clear then the PD process was successful and the patient is responding well. If the color is cloudy then, it requires physician's attention alerting about prevalent infections. Similarly if fibrin clots and/or blood clots are found then, it would call for attention for the physician and needs to be addressed with immediate effect.

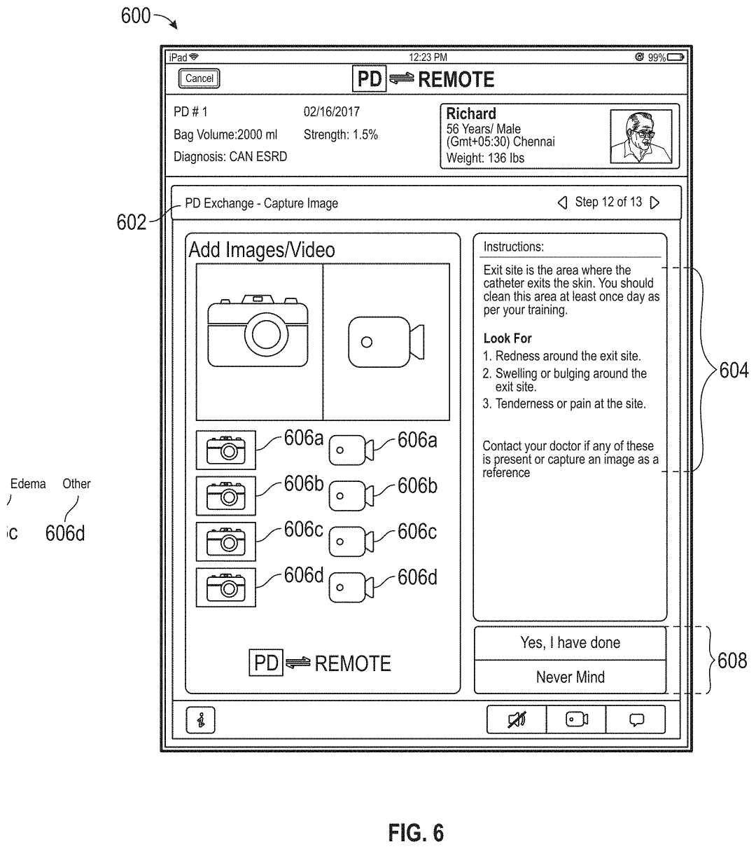

Referring to FIG. 6 is a screenshot 600, depicting the image upload option by the patient, according to an exemplary embodiment of the present disclosure. Exit site forms a crucial part of the PD system. Exit site may be defined as the area where the catheter exits the skin. The tutorial recommends the area to be cleansed once a day. Cleansing of the exit site is a necessity as upon infection it could lead to further complications in the PD treatment. A set of instructions 604 like look for redness around the exit site, swelling or bulging around the exit site, and tenderness or pain around the exit site along with an instruction to contact one's physician on the prevalence of any of these symptoms along with an image. The images and/or videos pertaining to those of exit site 606a, drain bag 606b, pedal edema 606c, and other 606d. Other images and/or videos may be inclusive of those images and/or videos other than exit site 606a, drain bag 606b, and pedal edema 606c. Once the instructions are taken and the patient and/or PD nurse is convinced to have followed all the steps, Yes, I have done option may be clicked and/or if the patient and/or PD nurse is used to these instructions then a never mind 608 option may be chosen. The images may be in high definition mode and for greater clarity zoom tool may be used in both images and video format where the video may be of short duration, not limiting to, 45 seconds. The images are configured to be horizontal images and/or vertical images. Horizontal images are those of various kinds of images like pedal edema, drain bag, exit site and the like. Whereas the vertical images tend to be uploaded if the physician needs to compare the images of first session, most commonly at the ‘break-in’ of PD with that of the second session and subsequent sessions. The images are configured to be archived, retrieved and utilised for research purposes. These images are horizontal when the break-in-date is depicted and the complete PD cycles done may be analysed, whereas the vertical ones are analysed date wise. Vertical images are loaded so that a date wise comparison of the patient progress may be achieved and easily comprehended by the PD nurse and/or the physician. The vertical images act as a base line comparator where for example: if the image was stored one year ago, then the picture may be analysed after a year to check the condition of the patient undergoing PD.

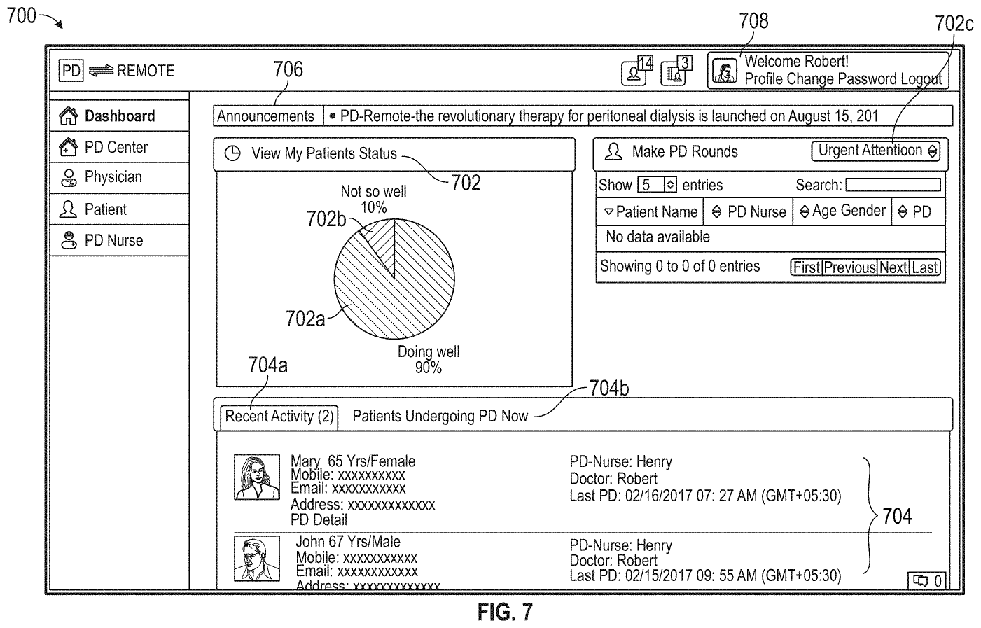

Referring to FIG. 7 is a screenshot 700, depicting the physician's dashboard reflecting the wellness pie chart, according to an exemplary embodiment of the present disclosure. The physician is in contact with the patient and/or PD nurse when the patient is being initiated for a PD procedure. The physician is provided with a secure login ID and a PDID password. A virtual keyboard may be provided for assisting easy login by the physician. As discussed, the physician's login credentials are communicated via an e-mail. The dashboard comprises of a pie chart 702 which gives a holistic approach to the wellbeing of the patients from the overall list of patients undergoing PD under the same physician. The pie chart may be divided into colors each for, not limiting to, doing well 702a as green, not so well 702b as orange, and urgent attention needed 702c as red. For example: a cloudy drain bag would be reflected as red color alerting the physician that the patient requires immediate attention. An option to make PD rounds is for the patients requiring urgent attention 702c and is not limited to, a drop down format. The details of patient's name, PD nurse, age and gender of the patient needing PD round are reflected. A recent activity option 704a reflects the details of the patients who have undergone PD and are waiting for the next PD and patients who have logged into their respective systems and are undergoing PD now is reflected as 704b. In both 704a and 704b the details are inclusive of, name of the patient, contact number, mail ID, postal address, the name of the PD nurse, name of the physician under whose supervision the patient is undergoing PD, and the date and time when the last PD was performed. The pie chart 702 is obtained as a result of the overall quality of life survey which would be discussed subsequently. The physician is equipped to add and/or delete the list of patient, add and/or delete the list of PD nurse and the like. The physician through one's account dashboard is configured to assess and rate PD nurse's performance. Announcements 706 update the patient and/or PD nurse regarding an upcoming event and/or an update relevant to the patient undergoing peritoneal dialysis procedure. The name and image of the physician 708 in charge for the patient undergoing peritoneal dialysis is reflected in the dashboard. The patients who are not doing well may be inclusive of those who need urgent attention and those who have complications but may be treated with some time lag. For example: Constipation may be needing attention of the physician to be fixed. But, if constipation leads to blockage in the draining of the effluent, then this would call for urgent attention.

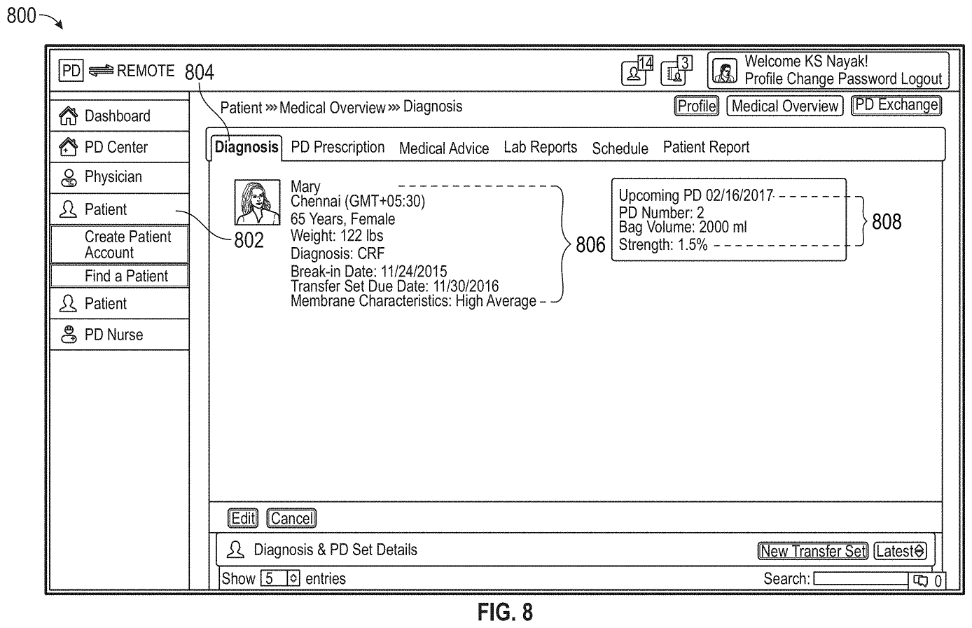

Referring to FIG. 8 is a screenshot 800, depicting the diagnosis page of the patient undergoing PD, according to an exemplary embodiment of the present disclosure. The patient 802 option enables the physician to visit the patient's diagnosis option 804. The details 806 reflected are inclusive of, but not limited to, name, Name of the patient, age and gender of the patient, body weight of the patient, the diagnosis [Chronic renal failure (CRF) in the figure] and the like. Further, a break-in-date is reflected which is the date from which the PD procedure had actually started for the patient under consideration. The body weight of the patient when measured between two PD episodes is expected to be same else, if an increase and/or an unusual decrease in weight would mean an immediate attention of the physician. A transfer set due date is also reflected. This helps the PD nurse and/or patient and/or the physician to note the time at which the transfer set needs to undergo replacement to avoid compromise with the PD process and results. For example: if the transfer set is due for replacement in four months the date would be reflected. The membrane characteristics option reflects the peritoneal membrane transport function in patients undergoing PD through an assessment test known as peritoneal equilibration test (PET). The membrane characteristics may be, not limited to, high, high-average, low-average, and low. The assessment is done through the transport rate of solutes such as, glucose, creatinine, and sodium. Further details of the patient 808 which is reflected may not be limited to, upcoming PD date, the number of PD to be performed, PD bag volume and strength of the peritoneal fluid.

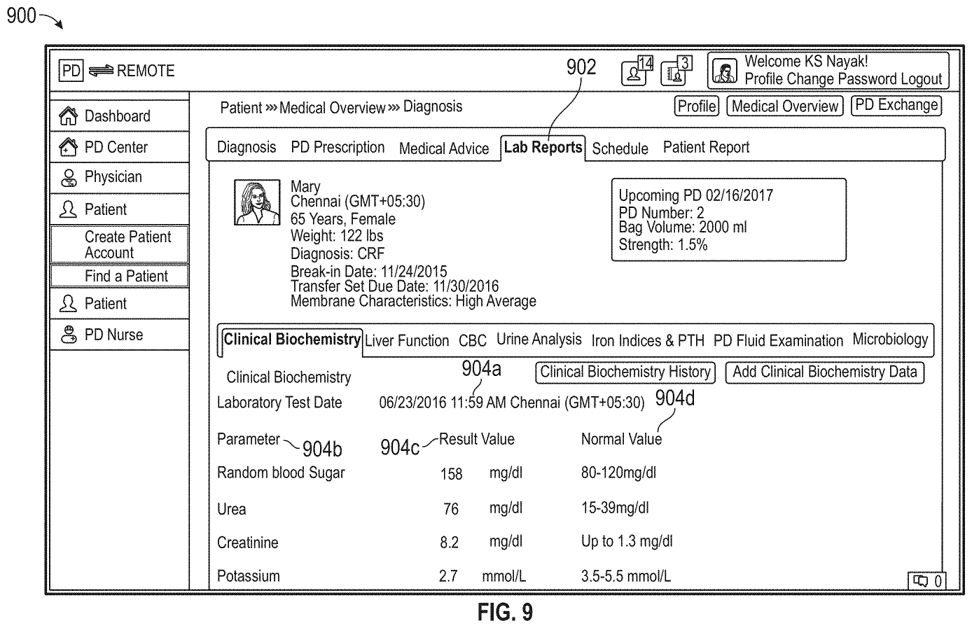

Referring to FIG. 9 is a screenshot 900, depicting the lab reports of the patient undergoing PD, according to an exemplary embodiment of the present disclosure. An option to have an access to detailed lab reports 902 of the patient enables the physician to decide the prescription and frame an opinion on the diagnosis. The basic details of the patient as discussed in FIG. 8 are reflected for easy identification of the patient by the physician. The lab reports may be inclusive of, but not limited to, clinical biochemistry, liver function including serum albumin, complete blood count (CBC), urine analysis, Iron indices and PTH, PD fluid examination, and Microbiology. Clinical biochemistry history and additional clinical biochemistry data may also be uploaded by the patient. For example: Clinical biochemistry may include, the date, time and place where the laboratory test was conducted 904a, the parameters which were tested 904b, the result value of the tested parameters 904c and the normal value 904d (which is the clinically acceptable limit) are reflected. The parameters 904b may not be limited to, random blood sugar, urea, creatinine, potassium and the like. Any aberration from the normal values would immediately alert the physician for changing the prescription and/or to act according to the immediate requirement of the patient undergoing PD. Post the lab reports option patient's nutritional status assessment and monitoring by the parameters, not limiting to, serum albumin, Bio impedance assessment (BIA), and anthropometry.

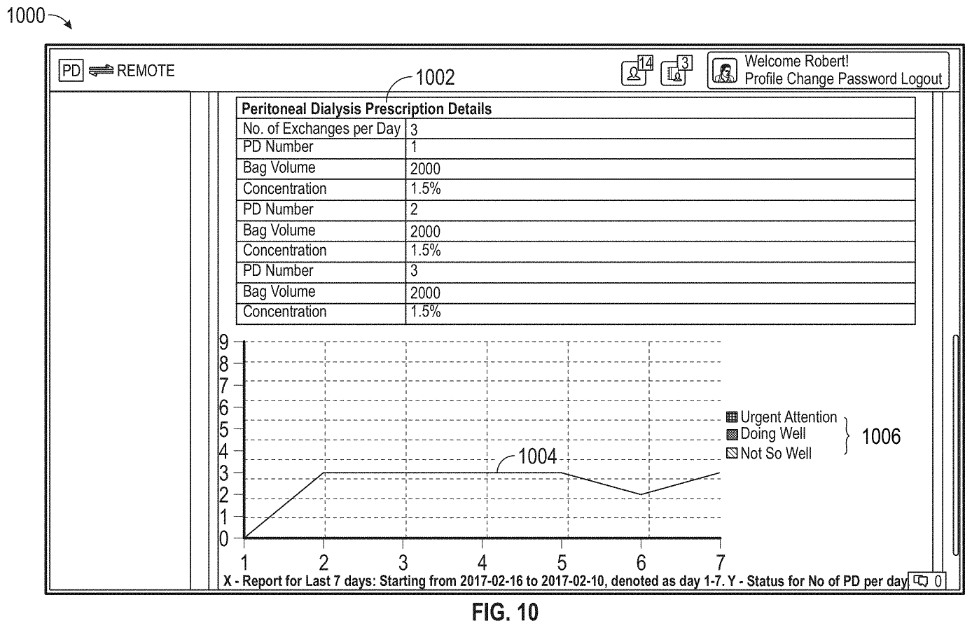

Referring to FIG. 10 is a screenshot 1000, depicting a graphical representation of prescription details of the patient undergoing PD, according to an exemplary embodiment of the present disclosure. The PD prescription details 1002 is a consolidated data of the PD's performed on a patient in a day and the corresponding parameters associated with it. For example the PD prescription details 1002 is for 3 exchanges per day, then the entire statistics may be represented in a tabular format below:

| | PD bag volume | Concentration (%) strength of the |
|---|---|---|
| PD NUMBER | (ml) | peritoneal fluid |
| 1 | 2000 | 1.5 |
| 2 | 2000 | 1.5 |
| 3 | 2000 | 1.5 |

Also, the prescription details would be inclusive of, any drug which needs to be administered for PD and/or otherwise. The above table may also be represented graphically 1004, where the color of the graphical line may be divided into colors each for, not limiting to, doing well as green, not so well as orange, and urgent attention needed as red, where the colors are represented as 1006. The X-axis may represent the PD reports of the last few days, not limiting to a week and the Y-axis may represent the status of number of PD performed on the patient. The linearity and uniformity in the graph would denote consistency in the patient's response to PD. Else; the physician may be alerted of an upcoming contingency. The above table may also be represented as a histogram. There may be a provision for setting an alarm based on the histogram obtained. The alarm may be set in a range. For example: an alarm range may be set for blood sugar levels ranging from 60 mg/dl to 140 mg/dl. If the histogram falls below and/or rises above the range then, an alarm sets off in the PD nurse's device and/or physician's device thus alerting them about the medical attention needed for the patient undergoing PD. Based on the histogram the prescription of the drugs may be changed.

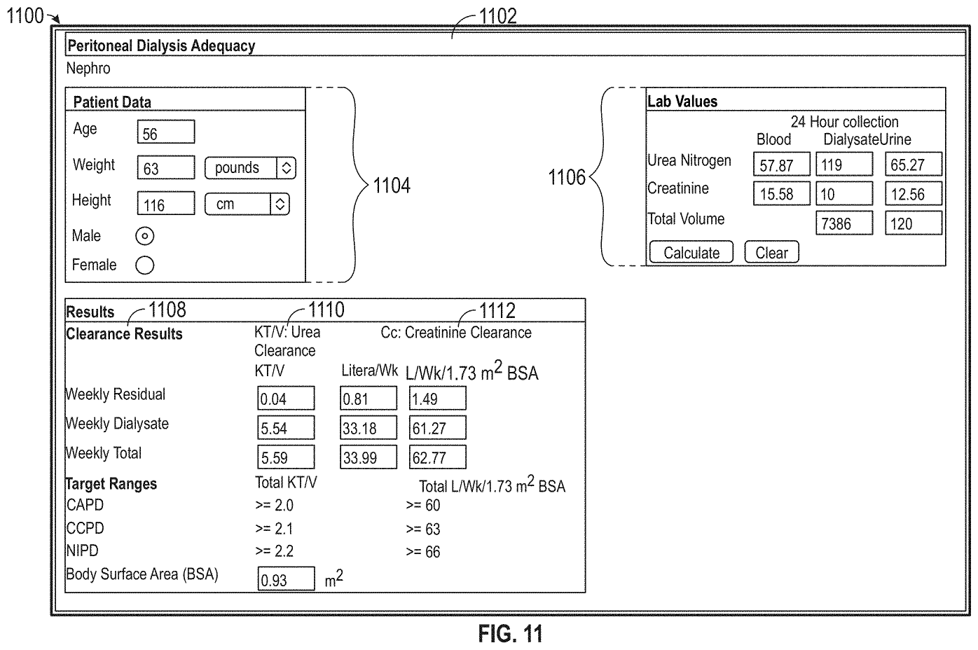

Referring to FIG. 11 is a screenshot 1100, depicting the adequacy of PD dose prescribed for the patient undergoing PD, according to an exemplary embodiment of the present disclosure. Adequacy may be defined as a combination of adequate fluid removal, adequate solute removal and adequate comfort in a patient undergoing PD accompanied by absence of uremic symptoms, stable blood pressure, and optimal fluid balance, management of anemia, metabolic control and optimal nutritional status. An adequate PD procedure is the one which provides a sufficient amount of dialysis treatment to maintain a uremic patient in the best condition and prevents complications due to uremic toxicity. The adequacy depends on two factors i.e. body surface area and residual renal function. The body surface area (BSA) may be measured by the following equation:

$$BSA = \frac{Body weight ( kg ) \times Height ( cm )}{3600}$$

Adequate PD may be assessed by considering the medical history, physical examination and laboratory tests of the patient undergoing PD. The medical history may take into account symptoms like presence and/or absence of, anorexia (loss of appetite), loss of taste, nausea, vomiting, insomnia, muscle weakness and itching. Physical examination may be inclusive of, but not limited to, blood pressure, nutritional assessment parameters and uremic odor.
Assessment of adequacy of PD may be done by the formula:

K t/V

Where: K=Clearance of urea
t=time
V=volume

and is recommended to be performed at an interval of 4 months.
K is affected by fill volume, surface area of the peritoneum, blood supply to the peritoneum and patient position

t is affected by dwell time, fill/drain times

V is affected by patient size
Creatinine clearance may be measured by: Considering Dialysate Creatinine Clearance+Renal Creatinine Clearance and also, taking into account the body surface area of the patient.

K t/V are believed to primarily target urea and creatinine levels in the blood post dialysis. In the screenshot 1100, the PD adequacy 1102 is measured upon considering the basic parameters of the patient 1104, not limiting to, age, weight in any acceptable metric system, height, gender, and the like. The lab values 1106 include, the parameters tested in the 24 hrs. collection period of the spent PD bags, where the parameters may not be limited to, urea in blood, urea in dialysate, and urea in urine. Similarly the parameters may also be inclusive of but not limited to, creatinine in blood, creatinine in dialysate, and creatinine in urine.

The clearance results 1108 may be those of weekly residual renal clearance, weekly dialysate, and weekly total clearances. The K t/V urea clearance 1110 and weekly creatinine clearance 1112 is also listed. The target ranges after CAPD (Continuous Ambulatory Peritoneal Dialysis), CCPD and NIPD (Nocturnal automated peritoneal dialysis) along with the body surface area is also disclosed. For example: if the weekly creatinine clearance value is less than 60 liters/week/1.73 m² BSA and it should ideally be equal to or more than 60 liters/week/1.73 m² BSA, then, the physician may alter the prescription for PD. Similar approach is applicable to the Kt/V urea clearances 1110. The adequacy of the PD would enable the physician to decide whether the dosage prescribed for the PD is sufficient for the patient or it does need some change.

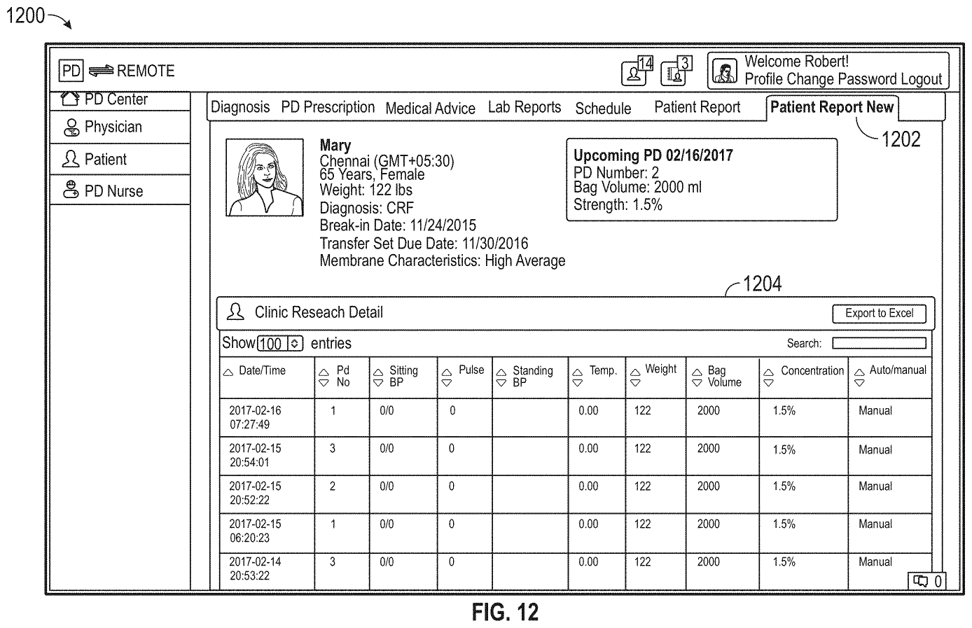

Referring to FIG. 12 is a screenshot 1200, depicting vital parameters of the patient undergoing PD, according to an exemplary embodiment of the present disclosure. The screenshot 1200, comprises of the patient's new report 1202 which would be inclusive of clinical research details 1204 like, date and time of PD which was conducted, the PD number, the pulse of the patient, the Blood pressure in both sitting and standing position, temperature of the body, PD bag volume, strength of the solution and whether the PD was automated and/or manual. These are reflected along with the basic details of the patient undergoing PD. The data is configured to be exported to excel sheet for easy reference of the physician. Alarms will be set within a specific range of values. For example, we set for blood sugar 60-200, if below 60 or above 200 alarms are set in the device. All the parameters will be in the histogram format. As another example if, the serum albumin levels (lower value is very important) if less than 3, alarm will be notified to PD nurse.

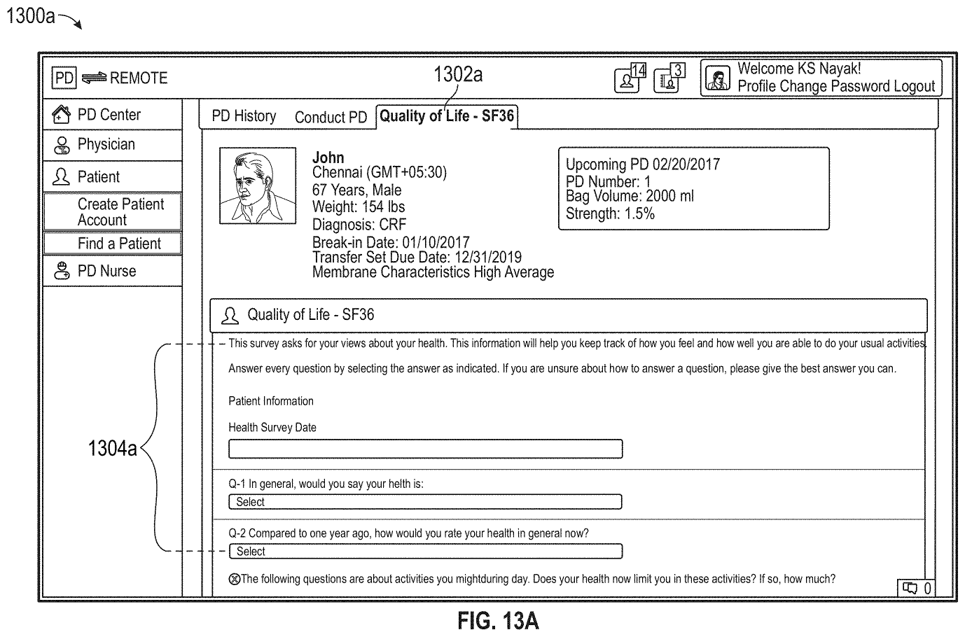

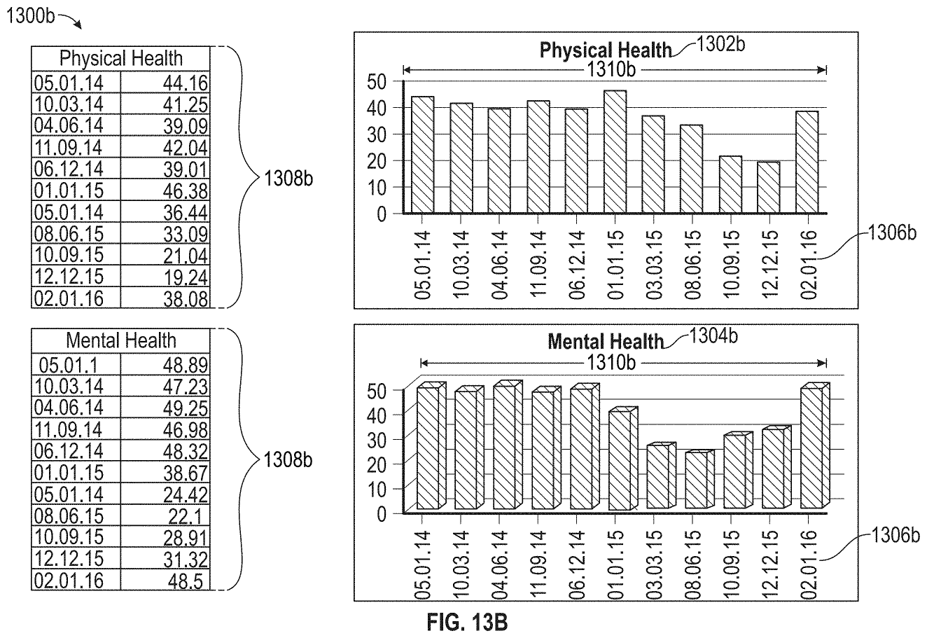

Referring to FIG. 13A is a screenshot 1300a, depicting the inputs regarding the quality of life by the patient undergoing PD. The quality of life -SF36 1302a is a patient self-reporting system utilized in a routinized basis by the physicians to anticipate as to how well the patient is able to conduct one's lifestyle and also to analyze the care outcomes in a patient. A set of self-reporting questionnaire 1304a is to be answered by the patient. For example: the set of questions may include, but not limited to:
a. In general, would you say your health is?
b. Compared to one year ago, how would you rate your health in general now?
c. Does your health now limit you in these activities? If so, how much?
d. During the past 4 weeks, to what extent has your physical health or emotional problems interfered with your normal social activities with family, friends, neighbors, or groups?
e. How much bodily pain have you had during the past 4 weeks?
f. During the past 4 weeks, how much of the time has your physical health or emotional problems interfered with your social activities (like visiting with friends, relatives, etc.)?

The feedback of the quality of life SF36 questionnaire 1302a would be reflected as one of the parameter in the pie chart (702 of FIG. 7) as a part of patient's status view. The total score is calculated out of 100 which would determine the quality of life of the patient undergoing PD.

Referring to FIG. 13B is a screenshot 1300b, FIG. 13A is a screenshot depicting the inputs regarding the quality of life by the patient undergoing peritoneal dialysis as a histogram, according to an exemplary embodiment of the present disclosure. The self-reporting system as discussed in FIG. 13A is configured to receive the inputs by the patients for assessing, not limited to, the physical health and mental health of the patient undergoing PD. A date when the survey was conducted may be filled in by the patient. The entry may be filling in the questionnaire not limited to, physical health, emotional health, energy levels, well-being, social interactions and behavior, in case of an ailment and/or an injury and/or as a result of the dialysis procedure the intensity of pain experienced may also be recorded. The general health may also be assigned values. After having answered all the queries an overall score may be obtained for not limited to, the physical health and mental health of the patient undergoing PD. In FIG. 13B the physical health 1302b and mental health 1304b are depicted as a histogram. The dates on which the survey for the physical health 1302b and mental health 1304b was done is depicted on X-axis as 1306b and the corresponding values obtained on each of the dates are depicted as 1308b in figures and 1310b as a histogram. The histogram 1310b enables the physician to have a bird's eye view of the progress in both physical health and mental health parameters of the patient. The histogram based presentation gives a quick review to the physician about the patient's well being whether there is an improvement or deterioration after a change in habit.

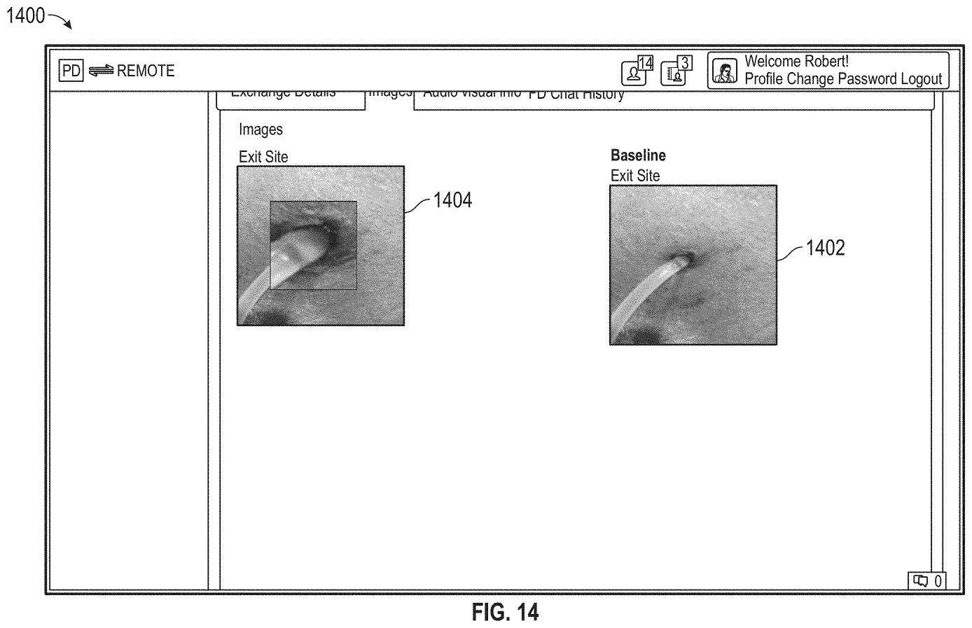

Referring to FIG. 14 is a screenshot 1400, depicting the exit site of the patient undergoing PD, according to an exemplary embodiment of the present disclosure. The exit site 1402 is the point from where the catheter exits the peritoneal cavity. The exit site 1402 is considered highly vulnerable to infections to the extent that the patient's own breath could be a leading cause of infection. A zoom tool 1404 may be used to have a zoomed view of the exit site 1402. Any amount of reddening of the skin at the exit site 1402, and any sign of infection would lead to further complications such as peritonitis and tunnel infection. (To be discussed in the later part of the description)

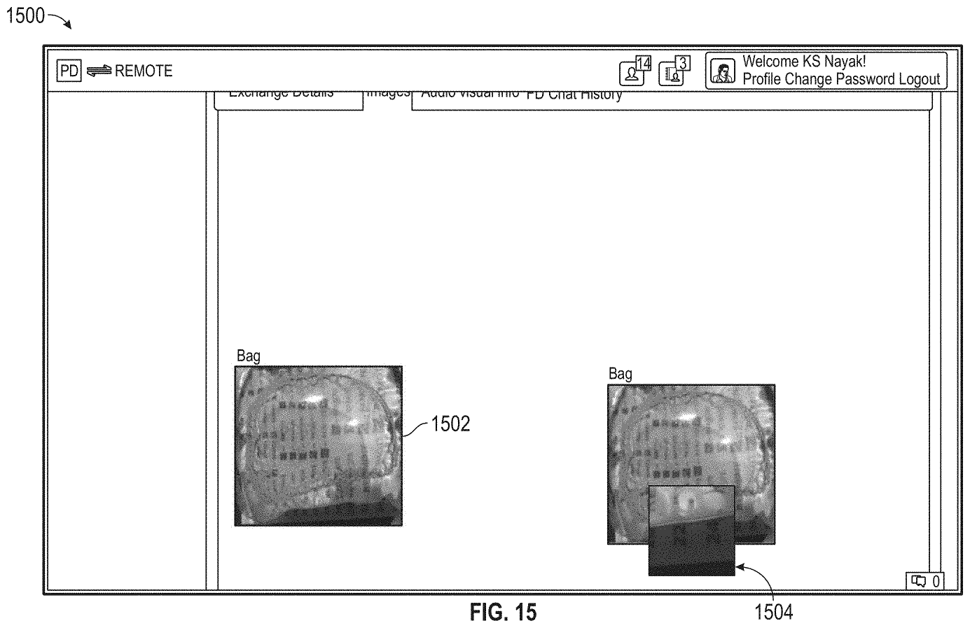

Referring to FIG. 15 is a screenshot 1500, depicting the usage of font sheet for the drain bag of the patient undergoing PD, according to an exemplary embodiment of the present disclosure. The clarity of the fluid in the drain bag 1502 may be measured using a font sheet 1504. The fonts may be ranging from not limited to, 14 to 64. For example: The drain bag after being filled with the fluid should exhibit clarity when viewed at font 14 and also when viewed at font 64. In case of the fonts not being visible at any numeral wound indicates peritonitis and call for immediate attention of the physician. For example the figure depicts the drain bag clarity at font 22 and 24.

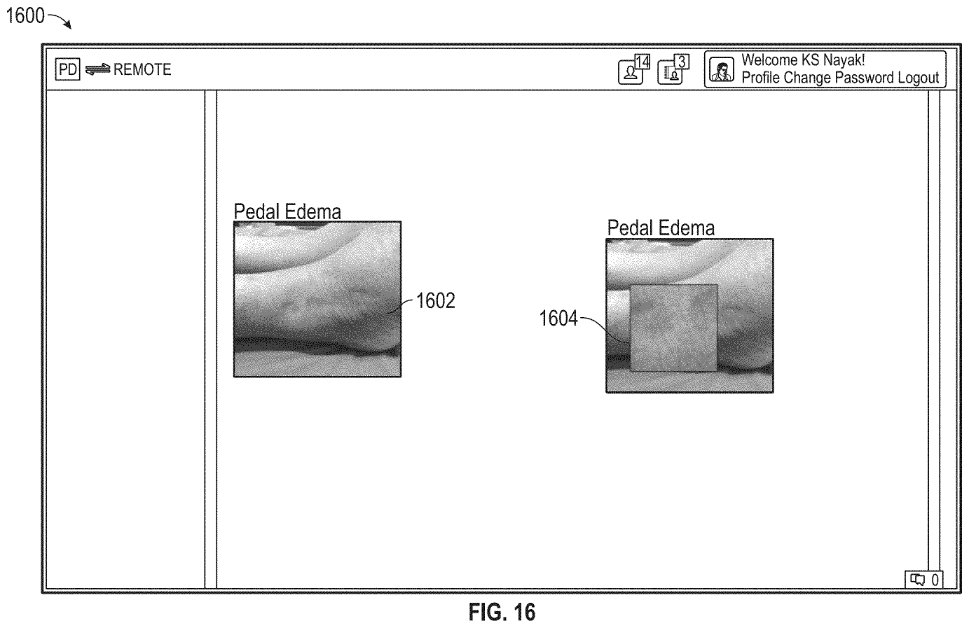

Referring to FIG. 16 is a screenshot 1600, depicting the usage of zoom tool for determining pedal edema, according to an exemplary embodiment of the present disclosure. Pedal edema is the retention of fluid especially in the ankle. This would normally indicate fluid retention in the patient undergoing PD. The picture of the ankle post dialysis 1602 may be observed with greater clarity using a zoom tool 1604 which depicts press marks on the ankle signifying water retention. Pedal edema is crucial for cardiac health of the patient. The higher the edema the greater is the risk on the cardiac health of the patient. The zoom tool functions to adjust the gain for optimal display of a condition to be studied. This would act as a pre-diagnostic implication for cardiovascular disease.

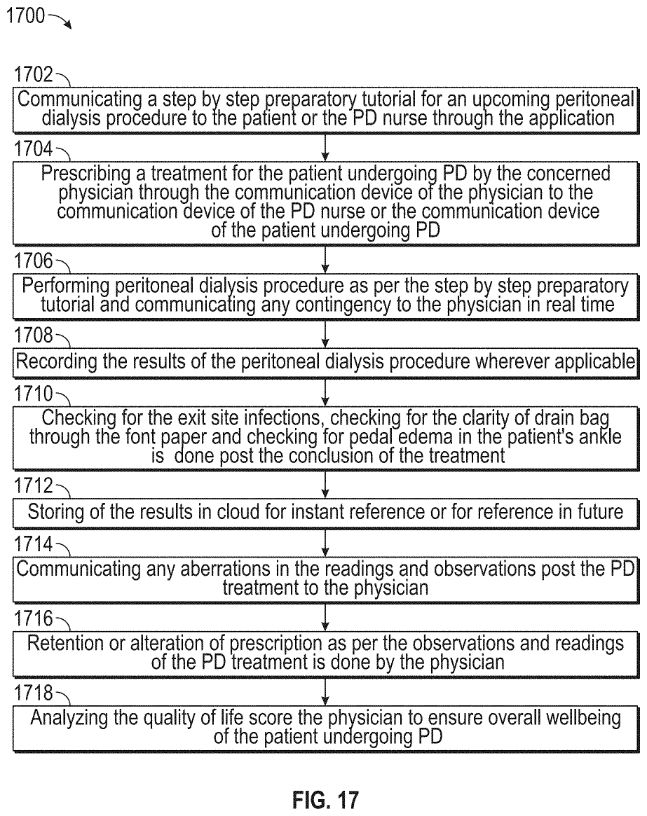

Referring to FIG. 17 is a flow chart 1700, depicting a method for remote monitoring and managing patients undergoing peritoneal dialysis, according to an exemplary embodiment of the present disclosure. The method starts at step 1702 by communicating a step by step preparatory tutorial for an upcoming peritoneal dialysis procedure to the patient or the PD nurse through the application. Further at step 1704, an advice is communicated for the patient undergoing PD by the concerned physician through the communication device of the physician to the communication device of the PD nurse or the communication device of the patient. Performing peritoneal dialysis procedure as per the step by step preparatory tutorial and communicating any contingency to the physician in real time is done at step 1706. Recording the results of the peritoneal dialysis procedure wherever applicable is done at step 1708. Checking for the exit site infections, checking for the clarity of drain bag through the font paper and checking for pedal edema in the patient's ankle is done post the conclusion of the treatment at step 1710. The results are stored in cloud for instant reference or for reference in future at step 1712. Communicating in real time any aberrations in the readings and observations post the PD treatment to the physician is done at step 1714. Retention or alteration of prescription as per the observations and readings of the PD treatment is done by the physician at step 1716. Finally, at step 1718 patient's quality of life score is analyzed by the physician to ensure overall wellbeing of the patient.

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

What is claimed is:

1. A method for remote monitoring and managing patients undergoing peritoneal dialysis comprising of:
establishing a communication link between at least one of: a peritoneal dialysis machine; a patient's display device; a PD nurse's display device and a physician's display device;
communicating a preparatory tutorial for an upcoming peritoneal dialysis procedure to at least one of: the patient; and the PD nurse through at least one of: the patient's display device; and the PD nurse's display device post authentication of the patient;
displaying at least one of: information regarding the type of peritoneal dialysis; a physiological data; and symptoms data of the patient undergoing peritoneal dialysis procedure on at least one of: the patient's display device; and the PD nurse's display device ;
communicating an advice for the patient undergoing peritoneal dialysis procedure by the concerned physician through the physician's display device to at least one of: the patient's display device; and the PD nurse's display device;
performing peritoneal dialysis procedure as per the preparatory tutorial and communicating any contingency to the physician in real time;
monitoring the parameters for analysing the peritoneal dialysis procedure;
recording the test results obtained during the peritoneal dialysis procedure wherever applicable and delivering the results obtained to the physician's display device;
storing the results obtained during the course of the peritoneal dialysis procedure in the cloud for at least one of: instant reference or for future use;
checking for at least one of: exit site infections; clarity of drain bag; and pedal edema post the conclusion of the peritoneal dialysis procedure by the physician; and
analysing of the observations and readings obtained post the peritoneal dialysis procedure and recommendation of prescriptions accordingly.

2. The method of claim 1, wherein the communication is between at least one of: a patient undergoing peritoneal dialysis procedure; a PD nurse assisting the patient undergoing peritoneal dialysis procedure; and a physician in charge for the patient undergoing peritoneal dialysis procedure.

3. The method of claim 1, wherein the communication is through at least one of: a plurality of text messages; plurality of videos; and a plurality of images.

4. The method of claim 1, wherein instructions for an upcoming peritoneal dialysis procedure is at least one of: the basic cleansing instructions on surrounding areas; organization of the essential equipment; setting up of the essential equipment; and keeping the vicinity immune to foreign bodies.

5. The method of claim 1, wherein the peritoneal dialysis procedure comprises the steps of: draining the waste fluid from the patient's body; flushing out the air bubbles in the transfer tube; and filling of the dialysis fluid in the patient's peritoneal cavity.

6. The method of claim 1, wherein the clarity of the drain bag is analyzed using a font paper placed under the drain bag.

7. The method of claim 1, wherein the parameters to be monitored are from at least one of: strength of the peritoneal dialysis solution; PD bag volume; drain weight; ultrafiltration; dwell time; and color of the drain liquid.

8. The method of claim 1, wherein the analysis of the observations and readings is performed by the assessment of at least one of: clinical research details; quality of life survey; peritoneal dialysis adequacy; and clinical biochemistry of the patient undergoing peritoneal dialysis procedure.

9. The method of claim 1, wherein the peritoneal dialysis adequacy is assessed by at least one of: Kt/V urea clearance; weekly creatinine clearance; and the peritoneal dialysis equilibration test.

10. The method of claim 1, wherein the prescription details is analyzed in at least one of: tabular form; a histogram; and a graphical form.

11. A system for remote monitoring and managing patients undergoing peritoneal dialysis comprising of;
a plurality of interconnected devices from at least one of: a patient's display device; a PD nurse's display device; and a physician's display device configured to communicate with each other and are further connected to a treatment device, whereby the treatment device is at least one of: a peritoneal dialysis machine; and a manual method of performing peritoneal dialysis;
a zoom tool embedded in the physician's display device configured to zoom in and zoom out at least one of: the plurality of videos; and a plurality of images;
a data processing architecture configured to perform at least one of: capture; process; and analyze at least one of: the patient's physiological data; and patient's symptoms data, whereby the patient's physiological data; and patient's symptoms data is displayed on the physician's display device; and
a cloud of network configured to communicate with at least one of: the patient's display device; the PD nurse's display device; and the physician's display device, whereby the cloud of network is configured to at least one of: store; recover; and back up the patient's physiological data; and patient's symptoms data.

12. The system of claim 1, wherein the communication between the pluralities of interconnected devices is configured to take place in real time.

13. The system of claim 1, wherein the assignment of at least one of: a scheduler; and a duty roster with respect to the plurality of patients under the purview of the particular PD nurse is allocated by at least one of: the PD nurse in charge of scheduling; and the physician with a predetermined calendar.
